(19) United States
(12) Patent Application Publication
(10) Pub. No.: US 2026/0108795 A1
(43) Pub. Date: Apr. 23, 2026

(54) SYSTEM

(71) Applicant: SoftBank Group Corp., Tokyo (JP)

(72) Inventor: Ryota NOMURA, Tokyo (JP)

(21) Appl. No.: 19/353,674

(22) Filed: Oct. 9, 2025

(30) Foreign Application Priority Data
Oct. 18, 2024 (JP) 2024-183582

(51) Int. Cl. A63B 71/06; A63B 24/00; G06T 7/20; G06V 40/16; G06V 40/20

(52) U.S. Cl. CPC A63B 71/0622; A63B 24/0006; A63B 24/0087; G06T 7/20; G06V 40/174; G06V 40/23; A63B 2024/0009; A63B 2024/0096; A63B 2071/0638; A63B 2220/806; G06T 2207/30201

(57) ABSTRACT

The system according to the embodiment includes a tracking unit, an analysis unit, and a feedback unit. The tracking unit tracks the user's motion. The analysis unit analyzes motion data tracked by the tracking unit. The feedback unit provides feedback based on data analyzed by the analysis unit.

### CROSS-REFERENCE TO RELATED APPLICATION

The present application claims priority to and incorporates by reference the entire contents of Japanese Patent Application No. 2024-183582 filed in Japan on Oct. 18, 2024.

### BACKGROUND OF THE INVENTION

### 1. Field of the Invention

The technology of this disclosure relates to a system.

### 2. Description of the Related Art

Japanese Patent Application Laid-open No. 2022-180282 discloses a persona chatbot control method executed by at least one processor, including: receiving a user utterance, adding the user utterance to a prompt containing instructions related to the character of the chatbot, encoding the prompt, inputting the encoded prompt into a language model, and generating a chatbot utterance in response to the user utterance.

In conventional technology, there has been a problem that it is difficult to provide real-time fitness form guidance, making it challenging to train with the correct form.

### SUMMARY OF THE INVENTION

The system according to the embodiment includes a tracking unit, an analysis unit, and a feedback unit. The tracking unit tracks the user's motion. The analysis unit analyzes motion data tracked by the tracking unit. The feedback unit provides feedback based on data analyzed by the analysis unit.

### BRIEF DESCRIPTION OF THE DRAWINGS

FIG. 1 is a conceptual diagram showing an example configuration of a data processing system according to the first embodiment;

FIG. 2 is a conceptual diagram showing an example of main functions of a data processing device and a smart device according to the first embodiment;

FIG. 3 is a conceptual diagram showing an example configuration of a data processing system according to the second embodiment;

FIG. 4 is a conceptual diagram showing an example of main functions of a data processing device and smart glasses according to the second embodiment;

FIG. 5 is a conceptual diagram showing an example configuration of a data processing system according to the third embodiment;

FIG. 6 is a conceptual diagram showing an example of main functions of a data processing device and a headset-type terminal according to the third embodiment;

FIG. 7 is a conceptual diagram showing an example configuration of a data processing system according to the fourth embodiment;

FIG. 8 is a conceptual diagram showing an example of main functions of a data processing device and a robot according to the fourth embodiment;

FIG. 9 shows an emotion map where multiple emotions are mapped; and

FIG. 10 shows an emotion map where multiple emotions are mapped.

### DETAILED DESCRIPTION OF THE PREFERRED EMBODIMENTS

Hereinafter, an example of an embodiment of the system related to the technology disclosed herein will be described with reference to the attached drawings.

First, the terminology used in the following description will be explained.

In the following embodiments, a processor with a sign (hereinafter simply referred to as “processor”) may be a single computing device or a combination of multiple computing devices. The processor may be a single type of computing device or a combination of multiple types of computing devices. Examples of computing devices include a CPU (Central Processing Unit), GPU (Graphics Processing Unit), GPGPU (General-Purpose computing on Graphics Processing Units), APU (Accelerated Processing Unit), or TPU (Tensor Processing Unit), among others.

In the following embodiments, a RAM (Random Access Memory) with a sign is a memory where information is temporarily stored and used as a work memory by the processor.

In the following embodiments, a storage with a sign is one or more non-volatile storage devices for storing various programs and parameters. Examples of non-volatile storage devices include flash memory (SSD (Solid State Drive)), magnetic disks (e.g., hard disks), or magnetic tapes, among others.

In the following embodiments, a communication I/F (Interface) with a sign is an interface including a communication processor and an antenna, among others. The communication I/F manages communication between multiple computers. Examples of communication standards applicable to the communication I/F include wireless communication standards such as 5G (5th Generation Mobile Communication System), Wi-Fi (registered trademark), or Bluetooth (registered trademark), among others.

In the following embodiments, “A and/or B” means “at least one of A and B.” In other words, “A and/or B” means it may be only A, only B, or a combination of A and B. Moreover, when expressing three or more items connected by “and/or,” the same concept as “A and/or B” applies.

### First Embodiment

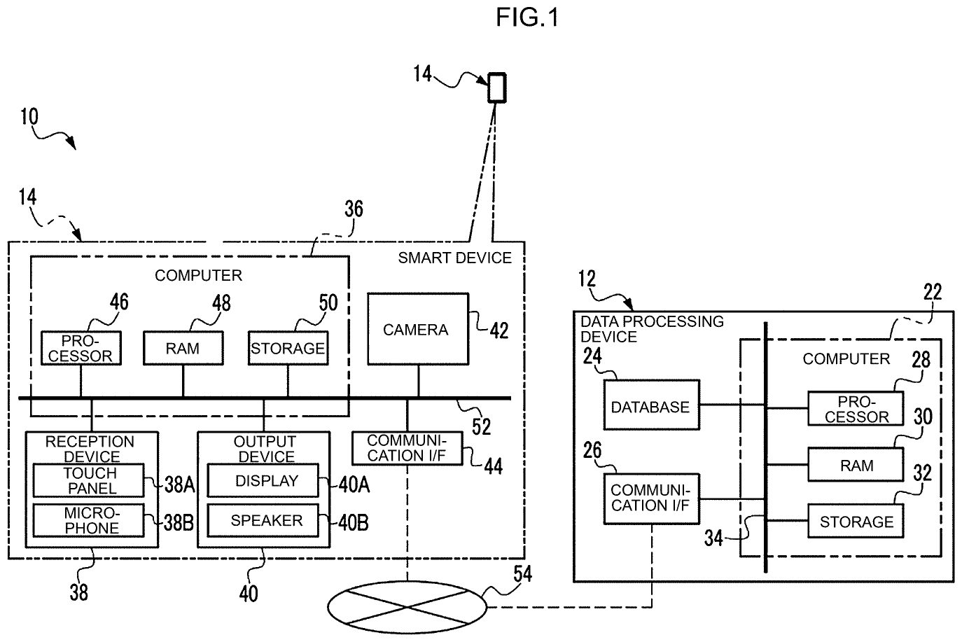

FIG. 1 shows an example configuration of a data processing system 10 according to the first embodiment.

As shown in FIG. 1, the data processing system 10 includes a data processing device 12 and a smart device 14. An example of the data processing device 12 is a server.

The data processing device 12 includes a computer 22, a database 24, and a communication I/F 26. The computer 22 includes a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN (Wide Area Network) and/or a LAN (Local Area Network), among others.

The smart device 14 includes a computer 36, a reception device 38, an output device 40, a camera 42, and a communication I/F 44. The computer 36 includes a processor 46, RAM 48, and storage 50. The processor 46, RAM 48, and storage 50 are connected to a bus 52. The reception device 38, output device 40, and camera 42 are also connected to the bus 52.

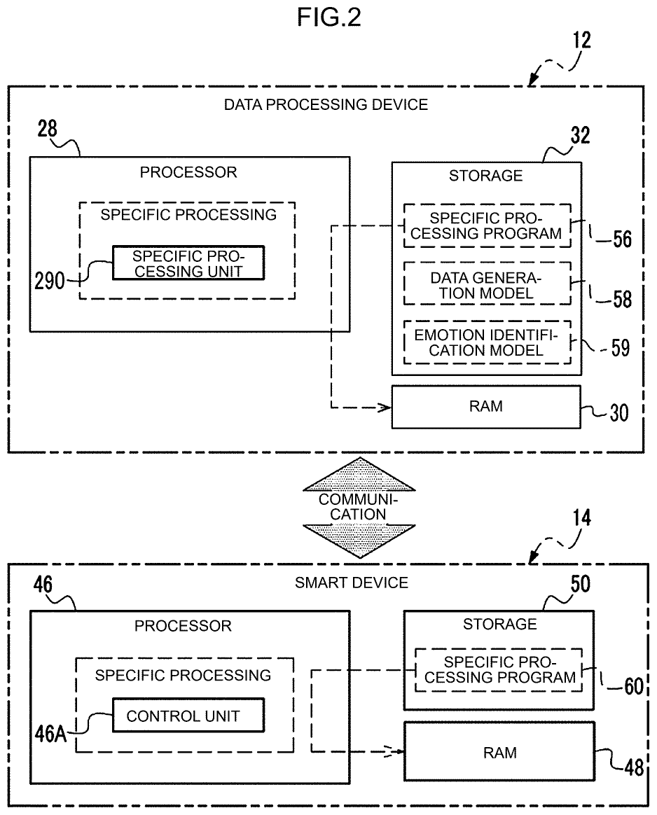

The reception device 38 includes a touch panel 38A and a microphone 38B, among others, and accepts user input. The touch panel 38A accepts user input by detecting contact from an indicating object (e.g., a pen or finger). The microphone 38B accepts user input by detecting the user's voice. The control unit 46A sends data indicating user input accepted by the touch panel 38A and microphone 38B to the data processing device 12. The data processing device 12 has a specific processing unit 290 (see FIG. 2) that acquires data indicating user input.

The output device 40 includes a display 40A and a speaker 40B, among others, and presents data to the user by outputting it in a perceptible form (e.g., audio and/or text). The display 40A displays visible information such as text and images according to instructions from the processor 46. The speaker 40B outputs audio according to instructions from the processor 46. The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors.

The communication I/F 44 is connected to the network 54. The communication I/F 44 and 26 manage the exchange of various information between the processor 46 and the processor 28 via the network 54.

FIG. 2 shows an example of the main functions of the data processing device 12 and the smart device 14.

As shown in FIG. 2, specific processing is performed in the data processing device 12 by the processor 28. The storage 32 stores a specific processing program 56. The specific processing program 56 is an example of a “program” related to the technology disclosed herein. The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart device 14, specific processing is performed by the processor 46. The storage 50 stores a specific processing program 60. The specific processing program 60 is used in conjunction with the specific processing program 56 by the data processing system 10. The processor 46 reads the specific processing program 60 from the storage 50 and executes it on the RAM 48. The specific processing is realized by the processor 46 operating as a control unit 46A according to the specific processing program 60 executed on the RAM 48. The smart device 14 may also have similar data generation models and emotion identification models as the data generation model 58 and emotion identification model 59, and perform the same processing as the specific processing unit 290 using these models.

Other devices besides the data processing device 12 may have the data generation model 58. For example, a server device (e.g., a generation server) may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.). Next, an example of processing by the data processing system 10 according to the first embodiment will be described.

### Example 1 of Embodiment

The fitness guide system according to the embodiment of the present invention is a system that utilizes multimodal generative AI and AR technology to provide real-time fitness form guidance. When a user trains at home or in a gym, the fitness guide system tracks the user's motion with a camera, and the generative AI analyzes and guides the form in real time. Since a trainer's avatar appearing through AR provides visual feedback of the correct form, the user can experience guidance similar to personal training. The fitness guide system provides support tailored to the needs of general consumers, fitness gyms, and personal trainers. Training with the correct form can reduce the risk of injury and maximize fitness effectiveness. For example, performing squats with the correct form can reduce the burden on the knees and lower back and effectively train muscle strength. Thus, the fitness guide system utilizing multimodal generative AI and AR technology is a highly beneficial tool for users. As a result, the fitness guide system enables users to train with the correct form by tracking, analyzing, and providing feedback on the user's motion in real time.

The fitness guide system according to the embodiment includes a tracking unit, an analysis unit, and a feedback unit. The tracking unit tracks the user's motion. For example, the tracking unit captures the user's motion in real time using a camera. The camera may include, for example, a high-resolution camera or a depth sensor. The tracking unit can accurately track the user's motion based on data obtained from the camera. The analysis unit analyzes motion data tracked by the tracking unit. For example, the analysis unit analyzes motion data using a generative AI and analyzes the user's form. The generative AI may be, for example, a text generative AI or a multimodal generative AI. The analysis unit compares the motion data with the correct form and identifies which parts are incorrect. The feedback unit provides feedback based on data analyzed by the analysis unit. For example, the feedback unit provides visual feedback of the correct form by displaying a trainer's avatar appearing through AR. The feedback unit can provide real-time guidance to the user. Thus, the fitness guide system according to the embodiment enables users to train with the correct form by tracking, analyzing, and providing feedback on the user's motion in real time.

The tracking unit tracks the user's motion. For example, the tracking unit captures the user's motion in real time using a camera. The camera may include, for example, a high-resolution camera or a depth sensor.

Specifically, the high-resolution camera can capture detailed user movements and facial expressions, and the depth sensor can accurately measure the user's position and distance. This allows the tracking unit to capture the user's motion in three dimensions and obtain more accurate data. Furthermore, by using multiple cameras in combination, the tracking unit can capture the user's motion from multiple angles and eliminate blind spots. For example, by placing cameras in front, on the side, and behind, the tracking unit can track the user's entire body in detail. In addition, the tracking unit can process data obtained from the camera in real time and immediately send the user's motion to the analysis unit. This enables real-time tracking of the user's motion and immediate feedback. Furthermore, the tracking unit can store the user's motion data and compare it with past data to understand the user's progress. As a result, the user can check the effectiveness of their training and maintain motivation.

The analysis unit analyzes motion data tracked by the tracking unit. For example, the analysis unit analyzes motion data using a generative AI and analyzes the user's form. The generative AI may be, for example, a text generative AI or a multimodal generative AI. Specifically, the generative AI receives the user's motion data as input and compares it with a reference database for the correct form. The generative AI analyzes the user's motion frame by frame and evaluates the accuracy of the motion in each frame. For example, when the user is performing a squat, the generative AI analyzes the knee angle, back position, foot placement, etc., and compares them with the correct form. The generative AI analyzes each element of the motion in detail and identifies which parts are incorrect. For example, it can detect problems such as the knees turning inward or the back rounding. Furthermore, the analysis unit can evaluate the improvement of the user's form by comparing it with past motion data. This allows the user to check changes in their form and feel the effects of training. The analysis unit can also propose individualized training plans based on the user's motion data. For example, if there are many problems with a particular motion, it can propose exercises to focus on improving that motion. Thus, the analysis unit can analyze the user's motion in detail and provide feedback tailored to individual needs.

The feedback unit provides feedback based on data analyzed by the analysis unit. For example, the feedback unit provides visual feedback of the correct form by displaying a trainer's avatar appearing through AR. Specifically, through an AR device worn by the user, the trainer's avatar appears in front of the user and demonstrates the correct form. The user can correct their own motion while watching the avatar's motion. Furthermore, the feedback unit can provide specific instructions through voice guidance or text messages. For example, it can provide real-time instructions such as “Please open your knees a little more” or “Keep your back straight.” This allows the user to train with more accurate form by combining visual feedback and voice guidance. In addition, the feedback unit can evaluate the user's training progress based on the user's motion data and provide feedback to increase motivation. For example, it can provide positive feedback such as “Your form has improved compared to last time” or “Great progress.” This allows the user to check their progress and maintain motivation for training. Furthermore, the feedback unit can collect user feedback and use it to improve the system. For example, the feedback unit can adjust the content and method of feedback based on user feedback to provide more effective guidance. Thus, the feedback unit can provide real-time guidance to the user and support training with the correct form.

The feedback unit can provide visual feedback of the correct form by displaying a trainer's avatar appearing through AR. For example, the feedback unit displays the trainer's avatar in front of the user and shows the correct form. The trainer's avatar can move in real time according to the user's motion. The feedback unit enables the user to intuitively understand the correct form. Thus, by providing visual feedback through AR, the user can intuitively understand the correct form. Some or all of the above-described processing in the feedback unit may be performed using AI or without using AI. For example, the feedback unit can input the motion of the trainer's avatar to a generative AI and have the generative AI execute the avatar's motion.

The tracking unit can capture the user's motion in real time using a camera. For example, the tracking unit captures the user's motion using a high-resolution camera. The tracking unit can accurately track the user's motion based on data obtained from the camera. The tracking unit can also capture the user's motion using a depth sensor, for example. Thus, by using a camera, the user's motion can be tracked accurately in real time. Some or all of the above-described processing in the tracking unit may be performed using AI or without using AI. For example, the tracking unit can input motion data obtained by the camera to a generative AI and have the generative AI analyze the motion data.

The analysis unit can compare the user's motion data with the correct form and identify which parts are incorrect. For example, the analysis unit analyzes motion data using a generative AI and analyzes the user's form. The generative AI may be, for example, a text generative AI or a multimodal generative AI. The analysis unit compares the motion data with the correct form and identifies which parts are incorrect. Thus, by comparing with the correct form, it becomes easier to identify errors in the user's motion. Some or all of the above-described processing in the analysis unit may be performed using AI or without using AI. For example, the analysis unit can input motion data to a generative AI and have the generative AI perform the comparison with the correct form.

The feedback unit can provide real-time guidance to the user based on the analysis results. For example, the feedback unit provides audio or visual feedback to the user. The feedback unit enables the user to intuitively understand the correct form. For example, the feedback unit displays the trainer's avatar in front of the user and shows the correct form. Thus, by providing real-time guidance, the user can immediately correct their form. Some or all of the above-described processing in the feedback unit may be performed using AI or without using AI. For example, the feedback unit can input the analysis results to a generative AI and have the generative AI provide real-time guidance.

The feedback unit can provide support tailored to the needs of general consumers, fitness gyms, and personal trainers. For example, the feedback unit provides simple feedback for general consumers and detailed feedback for fitness gyms. For personal trainers, the feedback unit can propose training plans and manage progress. Thus, by providing support tailored to the diverse needs of users, the system can accommodate a wide range of users. Some or all of the above-described processing in the feedback unit may be performed using AI or without using AI. For example, the feedback unit can input the user's needs to a generative AI and have the generative AI provide optimal feedback.

The tracking unit can optimize the tracking algorithm by referring to the user's past motion data during tracking. For example, the tracking unit adjusts the tracking algorithm based on data from the user's past training. The tracking unit learns specific motion patterns from the user's past motion data and improves tracking accuracy. The tracking unit customizes the tracking algorithm by referring to the user's past training history. Thus, by referring to past motion data, tracking accuracy can be improved. Some or all of the above-described processing in the tracking unit may be performed using AI or without using AI. For example, the tracking unit can input the user's past motion data to a generative AI and have the generative AI optimize the tracking algorithm.

The tracking unit can dynamically change tracking parameters during tracking according to the user's motion speed and rhythm. For example, when the user is performing fast movements, the tracking unit increases the tracking frame rate to improve accuracy. When the user is performing slow movements, the tracking unit lowers the tracking frame rate to save resources. The tracking unit adjusts tracking parameters in real time according to the user's motion rhythm. Thus, by adjusting tracking parameters according to motion speed and rhythm, more accurate tracking is possible. Some or all of the above-described processing in the tracking unit may be performed using AI or without using AI. For example, the tracking unit can input the user's motion speed and rhythm data to a generative AI and have the generative AI dynamically change the tracking parameters.

The tracking unit can analyze the user's environmental sounds during tracking and incorporate them as background information for the motion. For example, the tracking unit analyzes the user's surrounding environmental sounds to improve tracking accuracy. When the user is training while listening to music, the tracking unit adjusts tracking parameters according to the rhythm. The tracking unit analyzes the noise level around the user and optimizes tracking accuracy. Thus, by analyzing environmental sounds, tracking accuracy can be improved. Some or all of the above-described processing in the tracking unit may be performed using AI or without using AI. For example, the tracking unit can input the user's environmental sound data to a generative AI and have the generative AI analyze the environmental sounds.

The tracking unit can improve tracking accuracy during tracking by considering the influence of the user's clothing and accessories. For example, the tracking unit analyzes the color and pattern of the user's clothing to improve tracking accuracy. When the user is wearing accessories, the tracking unit adjusts tracking parameters considering their influence. The tracking unit analyzes the movement of the user's clothing and accessories and optimizes tracking accuracy. Thus, by considering the influence of clothing and accessories, tracking accuracy can be improved. Some or all of the above-described processing in the tracking unit may be performed using AI or without using AI. For example, the tracking unit can input the user's clothing and accessory data to a generative AI and have the generative AI improve tracking accuracy.

The analysis unit can improve analysis accuracy during analysis by referring to the user's past training data. For example, the analysis unit adjusts the analysis algorithm based on data from the user's past training. The analysis unit learns specific motion patterns from the user's past training data and improves analysis accuracy. The analysis unit customizes the analysis algorithm by referring to the user's past training history. Thus, by referring to past training data, analysis accuracy can be improved. Some or all of the above-described processing in the analysis unit may be performed using AI or without using AI. For example, the analysis unit can input the user's past training data to a generative AI and have the generative AI improve analysis accuracy.

The analysis unit can customize analysis criteria during analysis according to the user's body type and muscle mass. For example, the analysis unit adjusts analysis criteria based on the user's body type data. The analysis unit customizes analysis criteria considering the user's muscle mass. The analysis unit optimizes the analysis algorithm according to the user's body type and muscle mass. Thus, by customizing analysis criteria according to body type and muscle mass, more accurate analysis is possible. Some or all of the above-described processing in the analysis unit may be performed using AI or without using AI. For example, the analysis unit can input the user's body type and muscle mass data to a generative AI and have the generative AI customize the analysis criteria.

The analysis unit can determine the analysis priority during analysis based on the user's training goals. For example, if the user's training goal is to increase muscle strength, the analysis unit prioritizes the analysis of data related to muscle strength. If the user's training goal is weight loss, the analysis unit prioritizes the analysis of data related to calorie consumption. If the user's training goal is to improve flexibility, the analysis unit prioritizes the analysis of data related to flexibility. Thus, by determining the analysis priority based on training goals, more effective feedback can be provided. Some or all of the above-described processing in the analysis unit may be performed using AI or without using AI. For example, the analysis unit can input the user's training goal data to a generative AI and have the generative AI determine the analysis priority.

The analysis unit can improve analysis accuracy during analysis by referring to the user's diet and sleep data. For example, the analysis unit adjusts the analysis algorithm based on the user's diet data. The analysis unit improves analysis accuracy by considering the user's sleep data. The analysis unit optimizes the analysis algorithm by referring to the user's diet and sleep data. Thus, by referring to diet and sleep data, analysis accuracy can be improved. Some or all of the above-described processing in the analysis unit may be performed using AI or without using AI. For example, the analysis unit can input the user's diet and sleep data to a generative AI and have the generative AI improve analysis accuracy.

The feedback unit can select the optimal feedback method during feedback by referring to the user's past feedback history. For example, the feedback unit selects the optimal feedback method based on feedback the user has received in the past. The feedback unit learns specific feedback patterns from the user's past feedback history and provides the optimal method. The feedback unit customizes the feedback content by referring to the user's past feedback history. Thus, by referring to past feedback history, the optimal feedback method can be provided. Some or all of the above-described processing in the feedback unit may be performed using AI or without using AI. For example, the feedback unit can input the user's past feedback history to a generative AI and have the generative AI select the optimal feedback method.

The feedback unit can adjust the level of detail of the feedback during feedback according to the user's training progress. For example, if the user's training progress is good, the feedback unit provides detailed feedback. If the user's training progress is slow, the feedback unit provides concise feedback. The feedback unit customizes the feedback content according to the user's training progress. Thus, by adjusting the level of detail of the feedback according to training progress, more appropriate feedback can be provided. Some or all of the above-described processing in the feedback unit may be performed using AI or without using AI. For example, the feedback unit can input the user's training progress data to a generative AI and have the generative AI adjust the level of detail of the feedback.

The feedback unit can change the format of the feedback during feedback based on the user's training environment. For example, if the user is training at home, the feedback unit provides audio feedback. If the user is training at a gym, the feedback unit provides visual feedback. If the user is training outdoors, the feedback unit provides vibration feedback. Thus, by changing the format of the feedback based on the training environment, more appropriate feedback can be provided. Some or all of the above-described processing in the feedback unit may be performed using AI or without using AI. For example, the feedback unit can input the user's training environment data to a generative AI and have the generative AI change the format of the feedback.

The feedback unit can customize the feedback content during feedback by referring to the user's social media activity. For example, the feedback unit customizes the feedback content based on the training content the user has shared on social media. The feedback unit provides feedback by referring to advice from fitness influencers followed by the user on social media. The feedback unit provides feedback to increase training motivation based on the user's social media activity. Thus, by referring to social media activity, optimal feedback can be provided to the user. Some or all of the above-described processing in the feedback unit may be performed using AI or without using AI. For example, the feedback unit can input the user's social media activity data to a generative AI and have the generative AI customize the feedback content.

The system according to the embodiment is not limited to the above examples, and various modifications are possible, for example, as described below.

The fitness guide system may further include a heart rate monitoring unit configured to monitor the user's heart rate. The heart rate monitoring unit acquires the user's heart rate data in real time and sends it to the analysis unit. The analysis unit evaluates the user's exercise intensity based on the heart rate data and can recommend an appropriate training intensity. For example, if the user's heart rate is too high, the analysis unit instructs to lower the training intensity, and conversely, if the heart rate is too low, it instructs to increase the intensity. The analysis unit can also estimate the user's fatigue level based on the heart rate data and recommend a break if necessary. This allows the user to perform optimal training according to their physical condition.

The fitness guide system may further include a diet data acquisition unit configured to acquire the user's diet data. The diet data acquisition unit records the content of meals consumed by the user and sends it to the analysis unit. The analysis unit evaluates the user's nutritional status based on the diet data and can provide dietary advice to maximize training effectiveness. For example, if the user is lacking protein, the analysis unit recommends meals rich in protein, and conversely, if the user is consuming too many calories, it recommends calorie restriction. This allows the user to manage their health from both training and dietary perspectives.

The fitness guide system may further include a sleep data acquisition unit configured to acquire the user's sleep data. The sleep data acquisition unit records the user's sleep patterns and sends them to the analysis unit. The analysis unit evaluates the user's fatigue level based on the sleep data and can adjust the intensity and content of training. For example, if the user is not getting enough sleep, the analysis unit recommends lighter training, and conversely, if the user is getting enough sleep, it recommends more intense training. This allows the user to perform optimal training according to their sleep condition.

The fitness guide system may further include an environment monitoring unit configured to monitor the user's training environment. The environment monitoring unit acquires, in real time, the temperature, humidity, noise level, etc., of the user's training environment and sends them to the analysis unit. The analysis unit can propose optimal environmental conditions for training based on the environment data. For example, if the temperature is too high, the analysis unit instructs to suspend training, and conversely, if the temperature is appropriate, it instructs to continue training. This allows the user to train in an optimal environment.

The fitness guide system may further include a training plan optimization unit configured to optimize the training plan by referring to the user's training history. The training plan optimization unit proposes the optimal training plan based on data from the user's past training. For example, it can evaluate the effectiveness of past training and propose training that was highly effective again. It can also adjust to avoid training that the user struggled with in the past. This allows the user to execute the optimal training plan based on their training history. The following is a brief description of the processing flow of Example 1 of the Embodiment.
Step 1: The tracking unit tracks the user's motion. For example, the tracking unit captures the user's motion in real time using a camera. The camera may include, for example, a high-resolution camera or a depth sensor. The tracking unit can accurately track the user's motion based on data obtained from the camera.
Step 2: The analysis unit analyzes motion data tracked by the tracking unit. For example, the analysis unit analyzes motion data using a generative AI and analyzes the user's form. The generative AI may be, for example, a text generative AI or a multimodal generative AI. The analysis unit compares the motion data with the correct form and identifies which parts are incorrect.
Step 3: The feedback unit provides feedback based on data analyzed by the analysis unit. For example, the feedback unit provides visual feedback of the correct form by displaying a trainer's avatar appearing through AR. The feedback unit can provide real-time guidance to the user.

### Example 2 of Embodiment

The fitness guide system according to the embodiment of the present invention is a system that utilizes multimodal generative AI and AR technology to provide real-time fitness form guidance. When a user trains at home or in a gym, the fitness guide system tracks the user's motion with a camera, and the generative AI analyzes and guides the form in real time. Since a trainer's avatar appearing through AR provides visual feedback of the correct form, the user can experience guidance similar to personal training. The fitness guide system provides support tailored to the needs of general consumers, fitness gyms, and personal trainers. Training with the correct form can reduce the risk of injury and maximize fitness effectiveness. For example, performing squats with the correct form can reduce the burden on the knees and lower back and effectively train muscle strength. Thus, the fitness guide system utilizing multimodal generative AI and AR technology is a highly beneficial tool for users. As a result, the fitness guide system enables users to train with the correct form by tracking, analyzing, and providing feedback on the user's motion in real time.

The fitness guide system according to the embodiment includes a tracking unit, an analysis unit, and a feedback unit. The tracking unit tracks the user's motion. For example, the tracking unit captures the user's motion in real time using a camera. The camera may include, for example, a high-resolution camera or a depth sensor. The tracking unit can accurately track the user's motion based on data obtained from the camera. The analysis unit analyzes motion data tracked by the tracking unit. For example, the analysis unit analyzes motion data using a generative AI and analyzes the user's form. The generative AI may be, for example, a text generative AI or a multimodal generative AI. The analysis unit compares the motion data with the correct form and identifies which parts are incorrect. The feedback unit provides feedback based on data analyzed by the analysis unit. For example, the feedback unit provides visual feedback of the correct form by displaying a trainer's avatar appearing through AR. The feedback unit can provide real-time guidance to the user. Thus, the fitness guide system according to the embodiment enables users to train with the correct form by tracking, analyzing, and providing feedback on the user's motion in real time.

The tracking unit tracks the user's motion. For example, the tracking unit captures the user's motion in real time using a camera. The camera may include, for example, a high-resolution camera or a depth sensor. Specifically, the high-resolution camera can capture detailed user movements and facial expressions, and the depth sensor can accurately measure the user's position and distance. This allows the tracking unit to capture the user's motion in three dimensions and obtain more accurate data. Furthermore, by using multiple cameras in combination, the tracking unit can capture the user's motion from multiple angles and eliminate blind spots. For example, by placing cameras in front, on the side, and behind, the tracking unit can track the user's entire body in detail. In addition, the tracking unit can process data obtained from the camera in real time and immediately send the user's motion to the analysis unit. This enables real-time tracking of the user's motion and immediate feedback. Furthermore, the tracking unit can store the user's motion data and compare it with past data to understand the user's progress. As a result, the user can check the effectiveness of their training and maintain motivation.

The analysis unit analyzes motion data tracked by the tracking unit. For example, the analysis unit analyzes motion data using a generative AI and analyzes the user's form. The generative AI may be, for example, a text generative AI or a multimodal generative AI. Specifically, the generative AI receives the user's motion data as input and compares it with a reference database for the correct form. The generative AI analyzes the user's motion frame by frame and evaluates the accuracy of the motion in each frame. For example, when the user is performing a squat, the generative AI analyzes the knee angle, back position, foot placement, etc., and compares them with the correct form. The generative AI analyzes each element of the motion in detail and identifies which parts are incorrect. For example, it can detect problems such as the knees turning inward or the back rounding. Furthermore, the analysis unit can evaluate the improvement of the user's form by comparing it with past motion data. This allows the user to check changes in their form and feel the effects of training. The analysis unit can also propose individualized training plans based on the user's motion data. For example, if there are many problems with a particular motion, it can propose exercises to focus on improving that motion. Thus, the analysis unit can analyze the user's motion in detail and provide feedback tailored to individual needs.

The feedback unit provides feedback based on data analyzed by the analysis unit. For example, the feedback unit provides visual feedback of the correct form by displaying a trainer's avatar appearing through AR. Specifically, through an AR device worn by the user, the trainer's avatar appears in front of the user and demonstrates the correct form. The user can correct their own motion while watching the avatar's motion. Furthermore, the feedback unit can provide specific instructions through voice guidance or text messages. For example, it can provide real-time instructions such as “Please open your knees a little more” or “Keep your back straight.” This allows the user to train with more accurate form by combining visual feedback and voice guidance. In addition, the feedback unit can evaluate the user's training progress based on the user's motion data and provide feedback to increase motivation. For example, it can provide positive feedback such as “Your form has improved compared to last time” or “Great progress.” This allows the user to check their progress and maintain motivation for training. Furthermore, the feedback unit can collect user feedback and use it to improve the system. For example, the feedback unit can adjust the content and method of feedback based on user feedback to provide more effective guidance. Thus, the feedback unit can provide real-time guidance to the user and support training with the correct form.

The feedback unit can provide visual feedback of the correct form by displaying a trainer's avatar appearing through AR. For example, the feedback unit displays the trainer's avatar in front of the user and shows the correct form. The trainer's avatar can move in real time according to the user's motion. The feedback unit enables the user to intuitively understand the correct form. Thus, by providing visual feedback through AR, the user can intuitively understand the correct form. Some or all of the above-described processing in the feedback unit may be performed using AI or without using AI. For example, the feedback unit can input the motion of the trainer's avatar to a generative AI and have the generative AI execute the avatar's motion.

The tracking unit can capture the user's motion in real time using a camera. For example, the tracking unit captures the user's motion using a high-resolution camera. The tracking unit can accurately track the user's motion based on data obtained from the camera. The tracking unit can also capture the user's motion using a depth sensor, for example. Thus, by using a camera, the user's motion can be tracked accurately in real time. Some or all of the above-described processing in the tracking unit may be performed using AI or without using AI. For example, the tracking unit can input motion data obtained by the camera to a generative AI and have the generative AI analyze the motion data.

The analysis unit can compare the user's motion data with the correct form and identify which parts are incorrect. For example, the analysis unit analyzes motion data using a generative AI and analyzes the user's form. The generative AI may be, for example, a text generative AI or a multimodal generative AI. The analysis unit compares the motion data with the correct form and identifies which parts are incorrect. Thus, by comparing with the correct form, it becomes easier to identify errors in the user's motion. Some or all of the above-described processing in the analysis unit may be performed using AI or without using AI. For example, the analysis unit can input motion data to a generative AI and have the generative AI perform the comparison with the correct form.

The feedback unit can provide real-time guidance to the user based on the analysis results. For example, the feedback unit provides audio or visual feedback to the user. The feedback unit enables the user to intuitively understand the correct form. For example, the feedback unit displays the trainer's avatar in front of the user and shows the correct form. Thus, by providing real-time guidance, the user can immediately correct their form. Some or all of the above-described processing in the feedback unit may be performed using AI or without using AI. For example, the feedback unit can input the analysis results to a generative AI and have the generative AI provide real-time guidance.

The feedback unit can provide support tailored to the needs of general consumers, fitness gyms, and personal trainers. For example, the feedback unit provides simple feedback for general consumers and detailed feedback for fitness gyms. For personal trainers, the feedback unit can propose training plans and manage progress. Thus, by providing support tailored to the diverse needs of users, the system can accommodate a wide range of users. Some or all of the above-described processing in the feedback unit may be performed using AI or without using AI. For example, the feedback unit can input the user's needs to a generative AI and have the generative AI provide optimal feedback.

The tracking unit can estimate the user's emotion and adjust the tracking accuracy based on the estimated emotion. For example, if the user is feeling stressed, the tracking unit increases the tracking accuracy to provide more detailed feedback. If the user is relaxed, the tracking unit slightly loosens the tracking accuracy to emphasize natural movement. If the user is focused, the tracking unit optimizes the tracking accuracy to provide the most effective feedback. Thus, by adjusting the tracking accuracy according to the user's emotion, more appropriate feedback can be provided. Emotion estimation is realized, for example, by using an emotion engine or a generative AI as an emotion estimation function. The generative AI may be a text generative AI (e.g., LLM) or a multimodal generative AI, but is not limited to these examples. Some or all of the above-described processing in the tracking unit may be performed using AI or without using AI. For example, the tracking unit can input the user's emotion data to a generative AI and have the generative AI adjust the tracking accuracy.

The tracking unit can optimize the tracking algorithm by referring to the user's past motion data during tracking. For example, the tracking unit adjusts the tracking algorithm based on data from the user's past training. The tracking unit learns specific motion patterns from the user's past motion data and improves tracking accuracy. The tracking unit customizes the tracking algorithm by referring to the user's past training history. Thus, by referring to past motion data, tracking accuracy can be improved. Some or all of the above-described processing in the tracking unit may be performed using AI or without using AI. For example, the tracking unit can input the user's past motion data to a generative AI and have the generative AI optimize the tracking algorithm.

The tracking unit can dynamically change tracking parameters during tracking according to the user's motion speed and rhythm. For example, when the user is performing fast movements, the tracking unit increases the tracking frame rate to improve accuracy. When the user is performing slow movements, the tracking unit lowers the tracking frame rate to save resources. The tracking unit adjusts tracking parameters in real time according to the user's motion rhythm. Thus, by adjusting tracking parameters according to motion speed and rhythm, more accurate tracking is possible. Some or all of the above-described processing in the tracking unit may be performed using AI or without using AI. For example, the tracking unit can input the user's motion speed and rhythm data to a generative AI and have the generative AI dynamically change the tracking parameters.

The tracking unit can estimate the user's emotion and adjust the tracking frequency based on the estimated emotion. For example, if the user is feeling stressed, the tracking unit increases the tracking frequency to provide more detailed feedback. If the user is relaxed, the tracking unit slightly loosens the tracking frequency to emphasize natural movement. If the user is focused, the tracking unit optimizes the tracking frequency to provide the most effective feedback. Thus, by adjusting the tracking frequency according to the user's emotion, more appropriate feedback can be provided. Emotion estimation is realized, for example, by using an emotion engine or a generative AI as an emotion estimation function. The generative AI may be a text generative AI (e.g., LLM) or a multimodal generative AI, but is not limited to these examples. Some or all of the above-described processing in the tracking unit may be performed using AI or without using AI. For example, the tracking unit can input the user's emotion data to a generative AI and have the generative AI adjust the tracking frequency.

The tracking unit can analyze the user's environmental sounds during tracking and incorporate them as background information for the motion. For example, the tracking unit analyzes the user's surrounding environmental sounds to improve tracking accuracy. When the user is training while listening to music, the tracking unit adjusts tracking parameters according to the rhythm. The tracking unit analyzes the noise level around the user and optimizes tracking accuracy. Thus, by analyzing environmental sounds, tracking accuracy can be improved. Some or all of the above-described processing in the tracking unit may be performed using AI or without using AI. For example, the tracking unit can input the user's environmental sound data to a generative AI and have the generative AI analyze the environmental sounds.

The tracking unit can improve tracking accuracy during tracking by considering the influence of the user's clothing and accessories. For example, the tracking unit analyzes the color and pattern of the user's clothing to improve tracking accuracy. When the user is wearing accessories, the tracking unit adjusts tracking parameters considering their influence. The tracking unit analyzes the movement of the user's clothing and accessories and optimizes tracking accuracy. Thus, by considering the influence of clothing and accessories, tracking accuracy can be improved. Some or all of the above-described processing in the tracking unit may be performed using AI or without using AI. For example, the tracking unit can input the user's clothing and accessory data to a generative AI and have the generative AI improve tracking accuracy.

The analysis unit can estimate the user's emotion and adjust the analysis algorithm based on the estimated emotion. For example, if the user is feeling stressed, the analysis unit finely tunes the analysis algorithm to provide detailed feedback. If the user is relaxed, the analysis unit slightly loosens the analysis algorithm to emphasize natural movement. If the user is focused, the analysis unit optimizes the analysis algorithm to provide the most effective feedback. Thus, by adjusting the analysis algorithm according to the user's emotion, more appropriate feedback can be provided. Emotion estimation is realized, for example, by using an emotion engine or a generative AI as an emotion estimation function. The generative AI may be a text generative AI (e.g., LLM) or a multimodal generative AI, but is not limited to these examples. Some or all of the above-described processing in the analysis unit may be performed using AI or without using AI. For example, the analysis unit can input the user's emotion data to a generative AI and have the generative AI adjust the analysis algorithm.

The analysis unit can improve analysis accuracy during analysis by referring to the user's past training data. For example, the analysis unit adjusts the analysis algorithm based on data from the user's past training. The analysis unit learns specific motion patterns from the user's past training data and improves analysis accuracy. The analysis unit customizes the analysis algorithm by referring to the user's past training history. Thus, by referring to past training data, analysis accuracy can be improved. Some or all of the above-described processing in the analysis unit may be performed using AI or without using AI. For example, the analysis unit can input the user's past training data to a generative AI and have the generative AI improve analysis accuracy.

The analysis unit can customize analysis criteria during analysis according to the user's body type and muscle mass. For example, the analysis unit adjusts analysis criteria based on the user's body type data. The analysis unit customizes analysis criteria considering the user's muscle mass. The analysis unit optimizes the analysis algorithm according to the user's body type and muscle mass. Thus, by customizing analysis criteria according to body type and muscle mass, more accurate analysis is possible. Some or all of the above-described processing in the analysis unit may be performed using AI or without using AI. For example, the analysis unit can input the user's body type and muscle mass data to a generative AI and have the generative AI customize the analysis criteria.

The analysis unit can estimate the user's emotion and adjust the display method of the analysis results based on the estimated emotion. For example, if the user is feeling stressed, the analysis unit provides a simple and highly visible display method. If the user is relaxed, the analysis unit provides a display method that includes detailed information. If the user is focused, the analysis unit provides a display method that highlights the key points. Thus, by adjusting the display method according to the user's emotion, more appropriate feedback can be provided. Emotion estimation is realized, for example, by using an emotion engine or a generative AI as an emotion estimation function. The generative AI may be a text generative AI (e.g., LLM) or a multimodal generative AI, but is not limited to these examples. Some or all of the above-described processing in the analysis unit may be performed using AI or without using AI. For example, the analysis unit can input the user's emotion data to a generative AI and have the generative AI adjust the display method of the analysis results.

The analysis unit can determine the analysis priority during analysis based on the user's training goals. For example, if the user's training goal is to increase muscle strength, the analysis unit prioritizes the analysis of data related to muscle strength. If the user's training goal is weight loss, the analysis unit prioritizes the analysis of data related to calorie consumption. If the user's training goal is to improve flexibility, the analysis unit prioritizes the analysis of data related to flexibility. Thus, by determining the analysis priority based on training goals, more effective feedback can be provided. Some or all of the above-described processing in the analysis unit may be performed using AI or without using AI. For example, the analysis unit can input the user's training goal data to a generative AI and have the generative AI determine the analysis priority.

The analysis unit can improve analysis accuracy during analysis by referring to the user's diet and sleep data. For example, the analysis unit adjusts the analysis algorithm based on the user's diet data. The analysis unit improves analysis accuracy by considering the user's sleep data. The analysis unit optimizes the analysis algorithm by referring to the user's diet and sleep data. Thus, by referring to diet and sleep data, analysis accuracy can be improved. Some or all of the above-described processing in the analysis unit may be performed using AI or without using AI. For example, the analysis unit can input the user's diet and sleep data to a generative AI and have the generative AI improve analysis accuracy.

The feedback unit can estimate the user's emotion and adjust the feedback content based on the estimated emotion. For example, if the user is feeling stressed, the feedback unit provides feedback in gentle words. If the user is relaxed, the feedback unit provides detailed feedback. If the user is focused, the feedback unit provides feedback that highlights the key points. Thus, by adjusting the feedback content according to the user's emotion, more appropriate feedback can be provided. Emotion estimation is realized, for example, by using an emotion engine or a generative AI as an emotion estimation function. The generative AI may be a text generative AI (e.g., LLM) or a multimodal generative AI, but is not limited to these examples. Some or all of the above-described processing in the feedback unit may be performed using AI or without using AI. For example, the feedback unit can input the user's emotion data to a generative AI and have the generative AI adjust the feedback content.

The feedback unit can select the optimal feedback method during feedback by referring to the user's past feedback history. For example, the feedback unit selects the optimal feedback method based on feedback the user has received in the past. The feedback unit learns specific feedback patterns from the user's past feedback history and provides the optimal method. The feedback unit customizes the feedback content by referring to the user's past feedback history. Thus, by referring to past feedback history, the optimal feedback method can be provided. Some or all of the above-described processing in the feedback unit may be performed using AI or without using AI. For example, the feedback unit can input the user's past feedback history to a generative AI and have the generative AI select the optimal feedback method.

The feedback unit can adjust the level of detail of the feedback during feedback according to the user's training progress. For example, if the user's training progress is good, the feedback unit provides detailed feedback. If the user's training progress is slow, the feedback unit provides concise feedback. The feedback unit customizes the feedback content according to the user's training progress. Thus, by adjusting the level of detail of the feedback according to training progress, more appropriate feedback can be provided. Some or all of the above-described processing in the feedback unit may be performed using AI or without using AI. For example, the feedback unit can input the user's training progress data to a generative AI and have the generative AI adjust the level of detail of the feedback.

The feedback unit can estimate the user's emotion and adjust the timing of the feedback based on the estimated emotion. For example, if the user is feeling stressed, the feedback unit delays the timing of the feedback and provides it when the user is relaxed. If the user is relaxed, the feedback unit advances the timing and provides immediate feedback. If the user is focused, the feedback unit optimizes the timing and provides feedback at the most effective moment. Thus, by adjusting the timing of the feedback according to the user's emotion, more effective feedback can be provided. Emotion estimation is realized, for example, by using an emotion engine or a generative AI as an emotion estimation function. The generative AI may be a text generative AI (e.g., LLM) or a multimodal generative AI, but is not limited to these examples. Some or all of the above-described processing in the feedback unit may be performed using AI or without using AI. For example, the feedback unit can input the user's emotion data to a generative AI and have the generative AI adjust the timing of the feedback.

The feedback unit can change the format of the feedback during feedback based on the user's training environment. For example, if the user is training at home, the feedback unit provides audio feedback. If the user is training at a gym, the feedback unit provides visual feedback. If the user is training outdoors, the feedback unit provides vibration feedback. Thus, by changing the format of the feedback based on the training environment, more appropriate feedback can be provided. Some or all of the above-described processing in the feedback unit may be performed using AI or without using AI. For example, the feedback unit can input the user's training environment data to a generative AI and have the generative AI change the format of the feedback.

The feedback unit can customize the feedback content during feedback by referring to the user's social media activity. For example, the feedback unit customizes the feedback content based on the training content the user has shared on social media. The feedback unit provides feedback by referring to advice from fitness influencers followed by the user on social media. The feedback unit provides feedback to increase training motivation based on the user's social media activity. Thus, by referring to social media activity, optimal feedback can be provided to the user. Some or all of the above-described processing in the feedback unit may be performed using AI or without using AI. For example, the feedback unit can input the user's social media activity data to a generative AI and have the generative AI customize the feedback content.

The system according to the embodiment is not limited to the above examples, and various modifications are possible, for example, as described below.

The fitness guide system may further include a heart rate monitoring unit configured to monitor the user's heart rate. The heart rate monitoring unit acquires the user's heart rate data in real time and sends it to the analysis unit. The analysis unit evaluates the user's exercise intensity based on the heart rate data and can recommend an appropriate training intensity. For example, if the user's heart rate is too high, the analysis unit instructs to lower the training intensity, and conversely, if the heart rate is too low, it instructs to increase the intensity. The analysis unit can also estimate the user's fatigue level based on the heart rate data and recommend a break if necessary. This allows the user to perform optimal training according to their physical condition.

The fitness guide system may further include a training plan customization unit configured to estimate the user's emotion and customize the training plan based on the estimated emotion. The training plan customization unit recommends relaxing training if the user is feeling stressed, and recommends more challenging training if the user is relaxed. In addition, if the user is focused, it can recommend training to maintain concentration. Thus, the system can provide the optimal training plan according to the u ser's emotional state.

The fitness guide system may further include a diet data acquisition unit configured to acquire the user's diet data. The diet data acquisition unit records the content of meals consumed by the user and sends it to the analysis unit. The analysis unit evaluates the user's nutritional status based on the diet data and can provide dietary advice to maximize training effectiveness. For example, if the user is lacking protein, the analysis unit recommends meals rich in protein, and conversely, if the user is consuming too many calories, it recommends calorie restriction. This allows the user to manage their health from both training and dietary perspectives.

The fitness guide system may further include a motivation enhancement unit configured to estimate the user's emotion and enhance training motivation based on the estimated emotion. The motivation enhancement unit provides encouraging messages if the user is feeling stressed, and provides feedback that gives a sense of achievement if the user is relaxed. In addition, if the user is focused, it can provide advice to maintain concentration. Thus, the system can provide appropriate motivation enhancement measures according to the user's emotional state.

The fitness guide system may further include a sleep data acquisition unit configured to acquire the user's sleep data. The sleep data acquisition unit records the user's sleep patterns and sends them to the analysis unit. The analysis unit evaluates the user's fatigue level based on the sleep data and can adjust the intensity and content of training. For example, if the user is not getting enough sleep, the analysis unit recommends lighter training, and conversely, if the user is getting enough sleep, it recommends more intense training. This allows the user to perform optimal training according to their sleep condition.

The fitness guide system may further include a progress evaluation unit configured to estimate the user's emotion and evaluate training progress based on the estimated emotion. The progress evaluation unit performs lenient progress evaluation if the user is feeling stressed, and provides detailed progress evaluation if the user is relaxed. In addition, if the user is focused, it can optimize progress evaluation and provide the most effective feedback. Thus, the system can perform appropriate progress evaluation according to the user's emotional state.

The fitness guide system may further include an environment monitoring unit configured to monitor the user's training environment. The environment monitoring unit acquires, in real time, the temperature, humidity, noise level, etc., of the user's training environment and sends them to the analysis unit. The analysis unit can propose optimal environmental conditions for training based on the environment data. For example, if the temperature is too high, the analysis unit instructs to suspend training, and conversely, if the temperature is appropriate, it instructs to continue training. This allows the user to train in an optimal environment.

The fitness guide system may further include a break timing adjustment unit configured to estimate the user's emotion and adjust the timing of training breaks based on the estimated emotion. The break timing adjustment unit instructs the user to take a break earlier if the user is feeling stressed, and delays the break if the user is relaxed. In addition, if the user is focused, it can instruct the user to take a break at the optimal timing. Thus, the system can provide appropriate break timing according to the user's emotional state.

The fitness guide system may further include a training plan optimization unit configured to optimize the training plan by referring to the user's training history. The training plan optimization unit proposes the optimal training plan based on data from the user's past training. For example, it can evaluate the effectiveness of past training and propose training that was highly effective again. It can also adjust to avoid training that the user struggled with in the past. This allows the user to execute the optimal training plan based on their training history.

The fitness guide system may further include a feedback content adjustment unit configured to estimate the user's emotion and adjust the feedback content of training based on the estimated emotion. The feedback content adjustment unit provides feedback in gentle words if the user is feeling stressed, and provides detailed feedback if the user is relaxed. In addition, if the user is focused, it can provide feedback that highlights the key points.

Thus, the system can provide appropriate feedback content according to the user's emotional state.

The following is a brief description of the processing flow of Example 2 of the Embodiment.
Step 1: The tracking unit tracks the user's motion. For example, the tracking unit captures the user's motion in real time using a camera. The camera may include, for example, a high-resolution camera or a depth sensor. The tracking unit can accurately track the user's motion based on data obtained from the camera.
Step 2: The analysis unit analyzes motion data tracked by the tracking unit. For example, the analysis unit analyzes motion data using a generative AI and analyzes the user's form. The generative AI may be, for example, a text generative AI or a multimodal generative AI. The analysis unit compares the motion data with the correct form and identifies which parts are incorrect.
Step 3: The feedback unit provides feedback based on data analyzed by the analysis unit. For example, the feedback unit provides visual feedback of the correct form by displaying a trainer's avatar appearing through AR. The feedback unit can provide real-time guidance to the user.

The specific processing unit 290 sends the results of specific processing to the smart device 14. In the smart device 14, the control unit 46A causes the output device 40 to output the results of specific processing. The microphone 38B acquires voice indicating user input in response to the results of specific processing. The control unit 46A sends the voice data indicating user input acquired by the microphone 38B to the data processing device 12. In the data processing device 12, the specific processing unit 290 acquires the voice data.

The data generation model 58 is a so-called generative AI (Artificial Intelligence). An example of the data generation model 58 is a generative AI such as ChatGPT (registered trademark) (Internet search <URL: https://openai.com/blog/chatgpt>). The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

Moreover, the processing by the data processing system 10 described above is executed by the specific processing unit 290 of the data processing device 12 or the control unit 46A of the smart device 14, but it may be executed by both the specific processing unit 290 of the data processing device 12 and the control unit 46A of the smart device 14. Additionally, the specific processing unit 290 of the data processing device 12 acquires or collects necessary information for processing from the smart device 14 or external devices, and the smart device 14 acquires or collects necessary information for processing from the data processing device 12 or external devices.

Each of the plurality of elements including the above-described tracking unit, analysis unit, and feedback unit is implemented, for example, in at least one of the smart device 14 and the data processing apparatus 12. For example, the tracking unit captures the user's motion in real time using the camera 42 of the smart device 14. The analysis unit is implemented, for example, by a specific processing unit 290 of the data processing apparatus 12, analyzes motion data using a generative AI, and analyzes the user's form. The feedback unit provides visual feedback of the correct form by displaying a trainer's avatar appearing through AR, for example, using the output device 40 of the smart device 14. The correspondence between each unit and the device or control unit is not limited to the above examples, and various modifications are possible.

### Second Embodiment

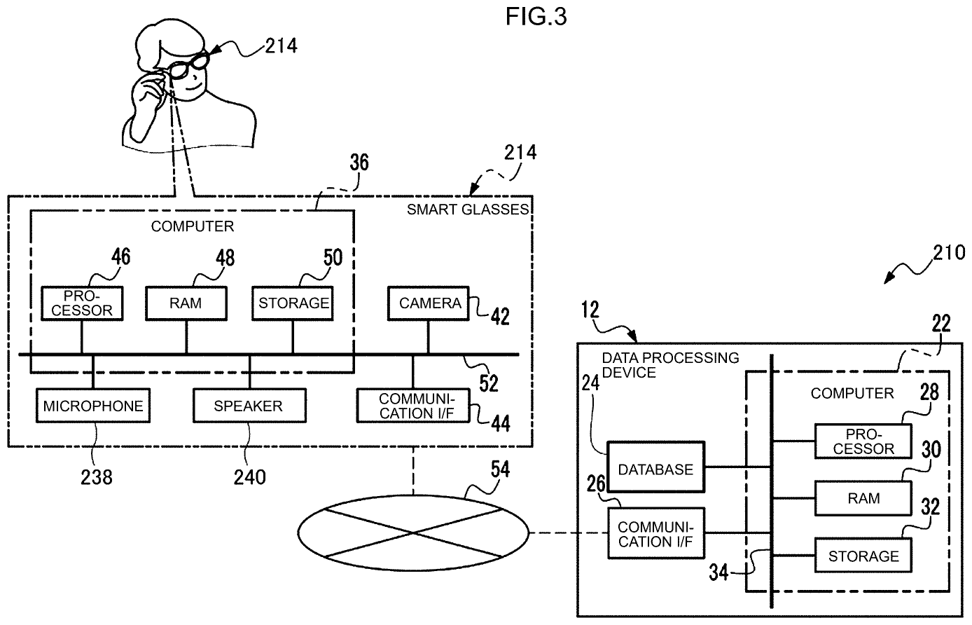

FIG. 3 shows an example configuration of a data processing system 210 according to the second embodiment.

As shown in FIG. 3, the data processing system 210 includes a data processing device 12 and smart glasses 214. An example of the data processing device 12 is a server.

The data processing device 12 includes a computer 22, a database 24, and a communication I/F 26. The computer 22 includes a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The smart glasses 214 includes a computer 36, a microphone 238, a speaker 240, a camera 42, and a communication I/F 44. The computer 36 includes a processor 46, RAM 48, and storage 50. The processor 46, RAM 48, and storage 50 are connected to a bus 52. The microphone 238, speaker 240, and camera 42 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

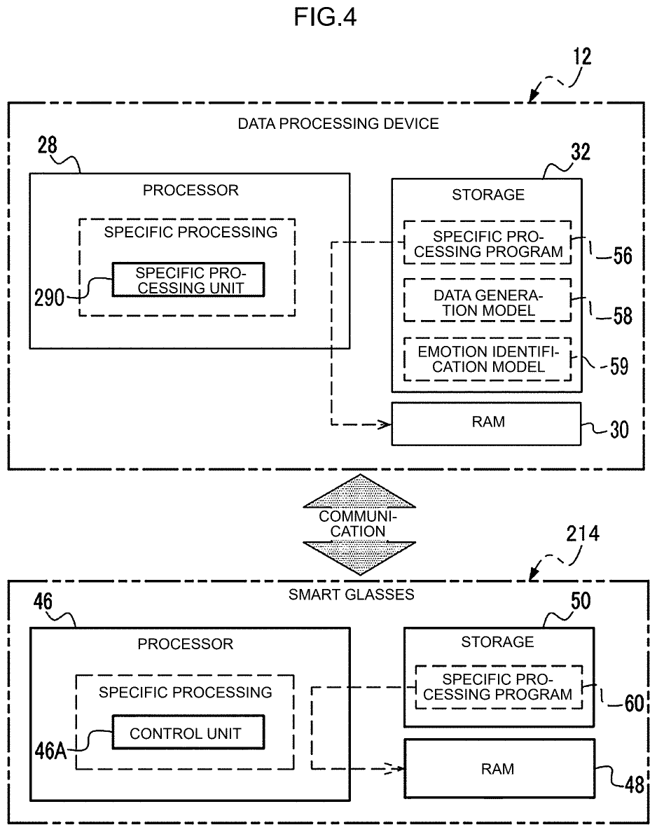

FIG. 4 shows an example of the main functions of the data processing device 12 and smart glasses 214. As shown in FIG. 4, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30. The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart glasses 214, specific processing is performed by the processor 46. The storage 50 stores a specific processing program 60. The processor 46 reads the specific processing program 60 from the storage 50 and executes it on the RAM 48. The specific processing is realized by the processor 46 operating as a control unit 46A according to the specific processing program 60 executed on the RAM 48. The smart glasses 214 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the smart glasses 214. In the smart glasses 214, the control unit 46A causes the speaker 240 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 210 according to the second embodiment performs the same processing as the data processing system 10 according to the first embodiment. The processing by the data processing system 210 is executed by the specific processing unit 290 of the data processing device 12 or the control unit 46A of the smart glasses 214, but it may be executed by both the specific processing unit 290 of the data processing device 12 and the control unit 46A of the smart glasses 214. Additionally, the specific processing unit 290 of the data processing device 12 acquires or collects necessary information for processing from the smart glasses 214 or external devices, and the smart glasses 214 acquires or collects necessary information for processing from the data processing device 12 or external devices.

Each of the plurality of elements including the above-described tracking unit, analysis unit, and feedback unit is implemented, for example, in at least one of the smart glasses 214 and the data processing apparatus 12. For example, the tracking unit captures the user's motion in real time using the camera 42 of the smart glasses 214. The analysis unit is implemented, for example, by a specific processing unit 290 of the data processing apparatus 12, analyzes motion data using a generative AI, and analyzes the user's form. The feedback unit provides visual feedback of the correct form by displaying a trainer's avatar appearing through AR, for example, using the speaker 240 of the smart glasses 214. The correspondence between each unit and the device or control unit is not limited to the above examples, and various modifications are possible.

### Third Embodiment

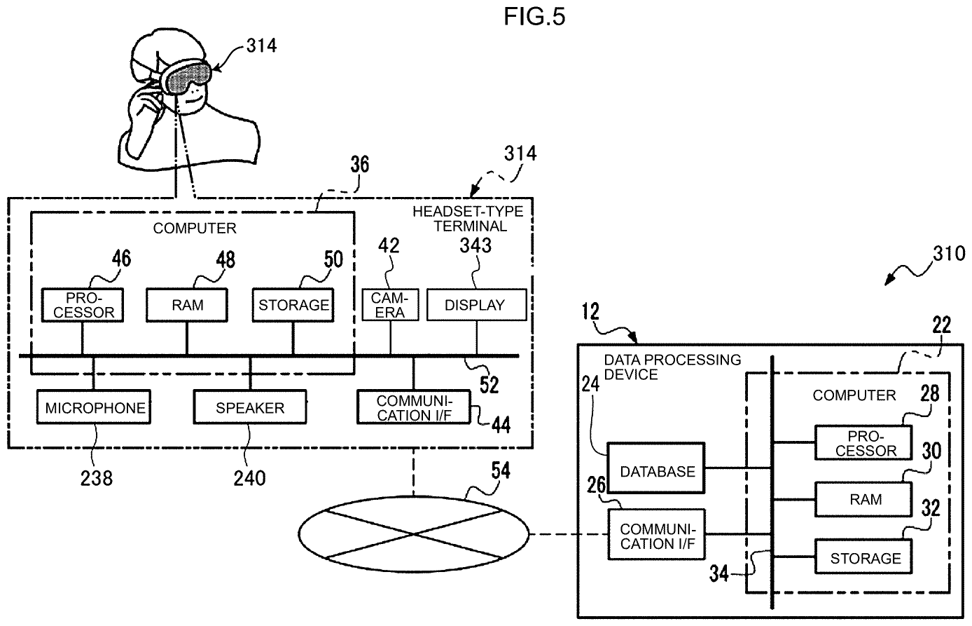

FIG. 5 shows an example configuration of a data processing system 310 according to the third embodiment.

As shown in FIG. 5, the data processing system 310 includes a data processing device 12 and a headset-type terminal 314. An example of the data processing device 12 is a server.

The data processing device 12 includes a computer 22, a database 24, and a communication I/F 26. The computer 22 includes a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The headset-type terminal 314 includes a computer 36, a microphone 238, a speaker 240, a camera 42, a communication I/F 44, and a display 343. The computer 36 includes a processor 46, RAM 48, and storage 50. The processor 46, RAM 48, and storage 50 are connected to a bus 52. The microphone 238, speaker 240, camera 42, and display 343 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

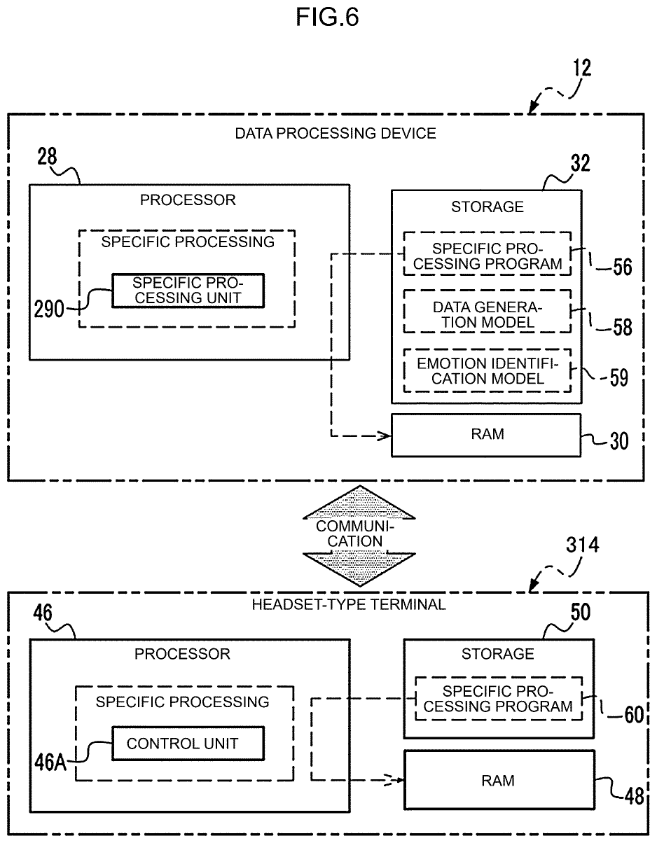

FIG. 6 shows an example of the main functions of the data processing device 12 and the headset-type terminal 314. As shown in FIG. 6, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the headset-type terminal 314, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The headset-type terminal 314 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the headset-type terminal 314. In the headset-type terminal 314, the control unit 46A causes the speaker 240 and the display 343 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 310 according to the third embodiment performs the same processing as the data processing system 10 according to the first embodiment. The processing by the data processing system 310 is executed by the specific processing unit 290 of the data processing device 12 or the control unit 46A of the headset-type terminal 314, but it may be executed by both the specific processing unit 290 of the data processing device 12 and the control unit 46A of the headset-type terminal 314. Additionally, the specific processing unit 290 of the data processing device 12 acquires or collects necessary information for processing from the headset-type terminal 314 or external devices, and the headset-type terminal 314 acquires or collects necessary information for processing from the data processing device 12 or external devices.

Each of the plurality of elements including the above-described tracking unit, analysis unit, and feedback unit is implemented, for example, in at least one of the headset-type terminal 314 and the data processing apparatus 12. For example, the tracking unit captures the user's motion in real time using the camera 42 of the headset-type terminal 314. The analysis unit is implemented, for example, by a specific processing unit 290 of the data processing apparatus 12, analyzes motion data using a generative AI, and analyzes the user's form. The feedback unit provides visual feedback of the correct form by displaying a trainer's avatar appearing through AR, for example, using the display 343 of the headset-type terminal 314. The correspondence between each unit and the device or control unit is not limited to the above examples, and various modifications are possible.

### Fourth Embodiment

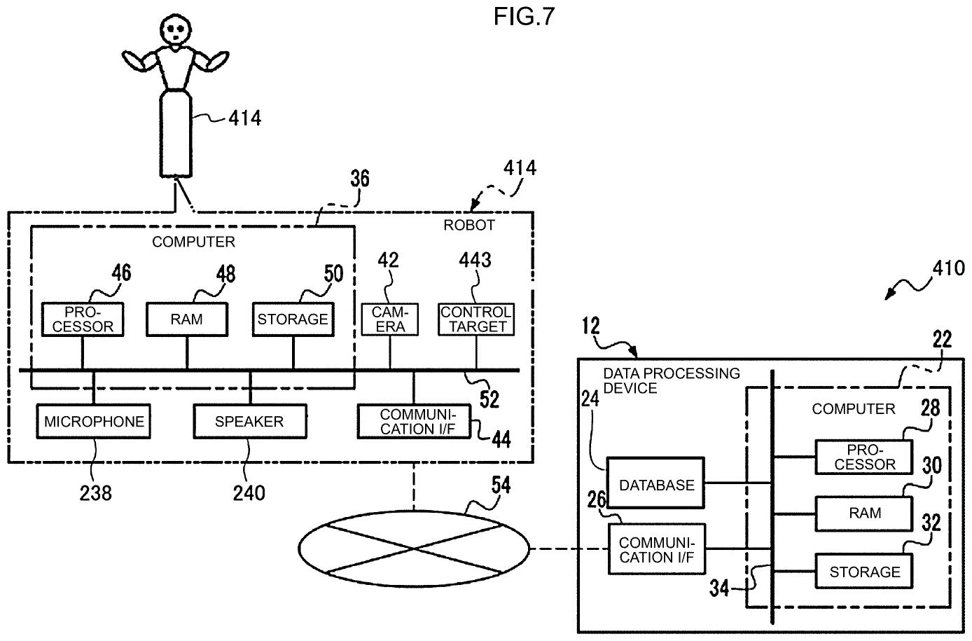

FIG. 7 shows an example configuration of a data processing system 410 according to the fourth embodiment.

As shown in FIG. 7, the data processing system 410 includes a data processing device 12 and a robot 414. An example of the data processing device 12 is a server.

The data processing device 12 includes a computer 22, a database 24, and a communication I/F 26. The computer 22 includes a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The robot 414 includes a computer 36, a microphone 238, a speaker 240, a camera 42, a communication I/F 44, and a control target 443. The computer 36 includes a processor 46, RAM 48, and storage 50. The processor 46, RAM 48, and storage 50 are connected to a bus 52. The microphone 238, speaker 240, camera 42, and control target 443 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS image sensors or CCD image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

The control target 443 includes a display device, LEDs for the eyes, and motors for driving arms, hands, and feet, among others. The posture and gestures of the robot 414 are controlled by controlling the motors for the arms, hands, and feet, among others. Some emotions of the robot 414 can be expressed by controlling these motors. Additionally, the expression of the robot 414 can be expressed by controlling the lighting state of the LEDs for the eyes of the robot 414.

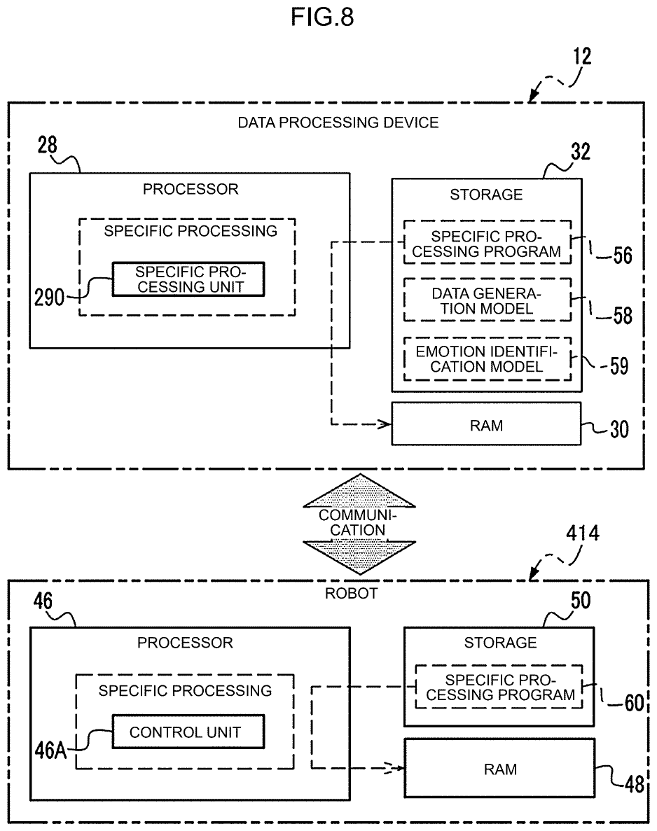

FIG. 8 shows an example of the main functions of the data processing device 12 and the robot 414. As shown in FIG. 8, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the robot 414, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The robot 414 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the robot 414. In the robot 414, the control unit 46A causes the speaker 240 and the control target 443 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 410 according to the fourth embodiment performs the same processing as the data processing system 10 according to the first embodiment. The processing by the data processing system 410 is executed by the specific processing unit 290 of the data processing device 12 or the control unit 46A of the robot 414, but it may be executed by both the specific processing unit 290 of the data processing device 12 and the control unit 46A of the robot 414. Additionally, the specific processing unit 290 of the data processing device 12 acquires or collects necessary information for processing from the robot 414 or external devices, and the robot 414 acquires or collects necessary information for processing from the data processing device 12 or external devices.

Each of the plurality of elements including the above-described tracking unit, analysis unit, and feedback unit is implemented, for example, in at least one of the robot 414 and the data processing apparatus 12. For example, the tracking unit captures the user's motion in real time using the camera 42 of the robot 414. The analysis unit is implemented, for example, by a specific processing unit 290 of the data processing apparatus 12, analyzes motion data using a generative AI, and analyzes the user's form. The feedback unit provides visual feedback of the correct form by displaying a trainer's avatar appearing through AR, for example, using the speaker 240 of the robot 414. The correspondence between each unit and the device or control unit is not limited to the above examples, and various modifications are possible.

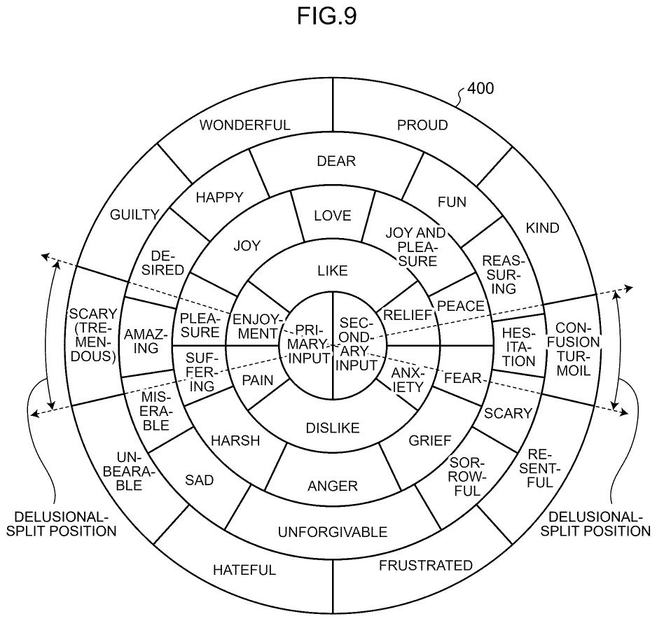

Note that the emotion identification model 59 as an emotion engine may determine the user's emotions according to a specific mapping. Specifically, the emotion identification model 59 may determine the user's emotions according to an emotion map, which is a specific mapping (see FIG. 9). Similarly, the emotion identification model 59 may determine the robot's emotions, and the specific processing unit 290 may perform specific processing using the robot's emotions.

FIG. 9 is a diagram showing an emotion map 400 where multiple emotions are mapped. In the emotion map 400, emotions are arranged concentrically radiating from the center. The closer to the center of the concentric circles, the more primitive the state of emotions is arranged. On the outer side of the concentric circles, emotions representing states and behaviors arising from mood are arranged. Emotions encompass concepts including emotional and mental states. On the left side of the concentric circles, emotions generally generated from reactions occurring in the brain are arranged. On the right side of the concentric circles, emotions generally induced by situational judgment are arranged. On the top and bottom of the concentric circles, emotions generated from reactions occurring in the brain and induced by situational judgment are arranged. Additionally, on the upper side of the concentric circles, “pleasant” emotions are arranged, and on the lower side, “unpleasant” emotions are arranged. In this way, in the emotion map 400, multiple emotions are mapped based on the structure from which emotions arise, and emotions that tend to occur simultaneously are mapped nearby.

These emotions are distributed in the 3 o'clock direction of the emotion map 400, and they usually move back and forth around reassurance and anxiety. In the right half of the emotion map 400, situational recognition takes precedence over internal sensations, giving a calm impression.

The inner side of the emotion map 400 represents the mind, and the outer side represents behavior, so the further out on the emotion map 400, the more visible (expressed in behavior) emotions become.

Here, human emotions are based on various balances like posture and blood sugar levels, and when these balances move away from the ideal, they indicate discomfort, and when they approach the ideal, they indicate comfort. In robots, cars, motorcycles, etc., emotions can be created based on various balances like posture and battery level, indicating discomfort when these balances move away from the ideal and comfort when they approach the ideal. The emotion map may be generated based on Dr. Mitsuyoshi's emotion map (Research on speech emotion recognition and brain physiological signal analysis systems related to emotions, Tokushima University, Doctoral dissertation: https://ci.nii.ac.jp/naid/500000375379). In the left half of the emotion map, emotions belonging to the domain called “reactions,” where sensations take precedence, are aligned. Additionally, in the right half of the emotion map, emotions belonging to the domain called “situations,” where situational recognition takes precedence, are aligned.

In the emotion map, two emotions that promote learning are defined. One is a negative emotion around “repentance” or “reflection” on the situation side. In other words, when a negative emotion arises in the robot, like “I never want to feel this way again” or “I don't want to be scolded again.” The other is an emotion around “desire” on the reaction side, which is positive. In other words, it is a positive feeling like “I want more” or “I want to know more.”

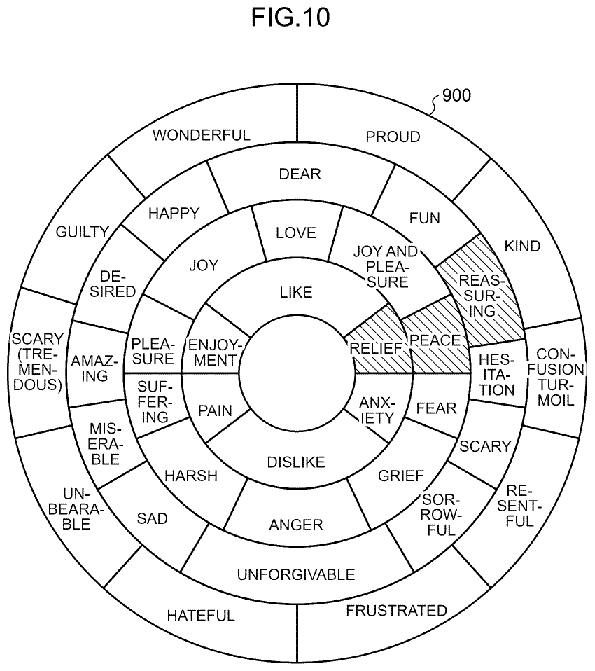

The emotion identification model 59 inputs user input into a pre-learned neural network, acquires emotion values indicating each emotion shown in the emotion map 400, and determines the user's emotions. This neural network is pre-learned based on multiple training data consisting of user input and combinations of emotion values indicating each emotion shown in the emotion map 400. Additionally, this neural network is learned so that emotions placed near each other in the emotion map 900 shown in FIG. 10 have similar values. FIG. 10 shows an example where multiple emotions like “reassured,” “calm,” and “confident” have similar emotion values.

In the above embodiments, an example form where specific processing is performed by a single computer 22 was described, but the technology disclosed herein is not limited to this, and distributed processing for specific processing by multiple computers including the computer 22 may be performed.

In the above embodiments, an example form where the specific processing program 56 is stored in the storage 32 was described, but the technology disclosed herein is not limited to this. For example, the specific processing program 56 may be stored in portable non-transitory storage media readable by a computer, such as a USB (Universal Serial Bus) memory. The specific processing program 56 stored in non-transitory storage media is installed in the computer 22 of the data processing device 12. The processor 28 executes specific processing according to the specific processing program 56.

Additionally, the specific processing program 56 may be stored in a storage device, such as a server connected to the data processing device 12 via the network 54, and downloaded and installed on the computer 22 in response to requests from the data processing device 12.

Furthermore, it is not necessary to store all of the specific processing program 56 in storage devices such as servers connected to the data processing device 12 via the network 54 or all in the storage 32, and a part of the specific processing program 56 may be stored.

Various processors, as shown next, can be used as hardware resources for executing specific processing. As processors, general-purpose processors that function as hardware resources for executing specific processing by executing software, i.e., programs, such as a CPU, can be mentioned. Additionally, as processors, dedicated electrical circuits with circuit configurations specially designed to execute specific processing, such as FPGA (Field-Programmable Gate Array), PLD (Programmable Logic Device), or ASIC (Application Specific Integrated Circuit), can be mentioned. Each processor has a built-in or connected memory, and each processor executes specific processing using the memory.

Hardware resources for executing specific processing may be composed of one of these various processors or a combination of two or more processors of the same or different types (e.g., a combination of multiple FPGAs or a combination of a CPU and FPGA). Additionally, hardware resources for executing specific processing may be a single processor.

As an example of composing with a single processor, firstly, there is a form where one or more CPUs and software are combined to constitute a single processor, which functions as hardware resources for executing specific processing. Secondly, there is a form using a processor, such as SoC (System-on-a-chip), that realizes the function of an entire system including multiple hardware resources for executing specific processing with a single IC chip. In this way, specific processing is realized using one or more of the various processors as hardware resources.

Furthermore, as a hardware structure of these various processors, more specifically, electrical circuits combined with circuit elements such as semiconductor elements can be used. Additionally, the specific processing described above is merely one example. Therefore, it goes without saying that unnecessary steps may be deleted, new steps may be added, or the order of processing may be changed within the scope not departing from the gist.

Additionally, in the examples described above, the explanation was divided into the first embodiment to the fourth embodiment, but parts or all of these embodiments may be combined. Additionally, the smart device 14, smart glasses 214, headset-type terminal 314, and robot 414 are examples, and each may be combined, or other devices may be used. Additionally, the examples described above were explained by dividing into form example 1 and form example 2, but these may be combined.

The descriptions and drawings shown above are detailed explanations of parts related to the technology disclosed herein and are merely examples of the technology disclosed herein. For example, the explanations regarding configurations, functions, actions, and effects above are explanations regarding examples of configurations, functions, actions, and effects of parts related to the technology disclosed herein. Therefore, it goes without saying that within the scope not departing from the gist of the technology disclosed herein, unnecessary parts may be deleted, new elements may be added, or replacements may be made to the descriptions and drawings shown above. Additionally, to avoid complexity and facilitate understanding of parts related to the technology disclosed herein, explanations concerning technical common knowledge and the like that do not require special explanation for enabling the implementation of the technology disclosed herein are omitted in the descriptions and drawings shown above.

All documents, patent applications, and technical standards described in this specification are incorporated by reference to the same extent as if each document, patent application, and technical standard were specifically and individually stated to be incorporated by reference in this specification.
[Additional Note 1] A system including: a tracking unit configured to track a user's motion; an analysis unit configured to analyze motion data tracked by the tracking unit; and a feedback unit configured to provide feedback based on data analyzed by the analysis unit.
[Additional Note 2] The system according to Additional Note 1, wherein the feedback unit provides visual feedback of the correct form by displaying a trainer's avatar appearing through AR.
[Additional Note 3] The system according to Additional Note 1, wherein the tracking unit captures the user's motion in real time using a camera.
[Additional Note 4] The system according to Additional Note 1, wherein the analysis unit compares the user's motion data with the correct form and identifies which parts are incorrect.
[Additional Note 5] The system according to Additional Note 1, wherein the feedback unit provides real-time guidance to the user based on the analysis results.
[Additional Note 6] The system according to Additional Note 1, wherein the feedback unit provides support tailored to the needs of general consumers, fitness gyms, and personal trainers.
[Additional Note 7] The system according to Additional Note 1, wherein the tracking unit estimates the user's emotion and adjusts the tracking accuracy based on the estimated emotion.
[Additional Note 8] The system according to Additional Note 1, wherein the tracking unit optimizes the tracking algorithm by referring to the user's past motion data during tracking.
[Additional Note 9] The system according to Additional Note 1, wherein the tracking unit dynamically changes tracking parameters during tracking according to the user's motion speed and rhythm.
[Additional Note 10] The system according to Additional Note 1, wherein the tracking unit estimates the user's emotion and adjusts the tracking frequency based on the estimated emotion.
[Additional Note 11] The system according to Additional Note 1, wherein the tracking unit analyzes the user's environmental sounds during tracking and incorporates them as background information for the motion.
[Additional Note 12] The system according to Additional Note 1, wherein the tracking unit improves tracking accuracy during tracking by considering the influence of the user's clothing and accessories.
[Additional Note 13] The system according to Additional Note 1, wherein the analysis unit estimates the user's emotion and adjusts the analysis algorithm based on the estimated emotion.
[Additional Note 14] The system according to Additional Note 1, wherein the analysis unit improves analysis accuracy during analysis by referring to the user's past training data.
[Additional Note 15] The system according to Additional Note 1, wherein the analysis unit customizes analysis criteria during analysis according to the user's body type and muscle mass.
[Additional Note 16] The system according to Additional Note 1, wherein the analysis unit estimates the user's emotion and adjusts the display method of the analysis results based on the estimated emotion.
[Additional Note 17] The system according to Additional Note 1, wherein the analysis unit determines the analysis priority during analysis based on the user's training goals.
[Additional Note 18] The system according to Additional Note 1, wherein the analysis unit improves analysis accuracy during analysis by referring to the user's diet and sleep data.
[Additional Note 19] The system according to Additional Note 1, wherein the feedback unit estimates the user's emotion and adjusts the feedback content based on the estimated emotion.
[Additional Note 20] The system according to Additional Note 1, wherein the feedback unit selects the optimal feedback method during feedback by referring to the user's past feedback history.
[Additional Note 21] The system according to Additional Note 1, wherein the feedback unit adjusts the level of detail of the feedback during feedback according to the user's training progress.
[Additional Note 22] The system according to Additional Note 1, wherein the feedback unit estimates the user's emotion and adjusts the timing of the feedback based on the estimated emotion.
[Additional Note 23] The system according to Additional Note 1, wherein the feedback unit changes the format of the feedback during feedback based on the user's training environment.
[Additional Note 24] The system according to Additional Note 1, wherein the feedback unit customizes the feedback content during feedback by referring to the user's social media activity.

What is claimed is:

1. A system comprising: a tracking unit configured to track a user's motion; an analysis unit configured to analyze motion data tracked by the tracking unit; and a feedback unit configured to provide feedback based on data analyzed by the analysis unit.

2. The system according to claim 1, wherein the feedback unit provides visual feedback of the correct form by displaying a trainer's avatar appearing through AR.

3. The system according to claim 1, wherein the tracking unit captures the user's motion in real time using a camera.

4. The system according to claim 1, wherein the analysis unit compares the user's motion data with the correct form and identifies which parts are incorrect.

5. The system according to claim 1, wherein the feedback unit provides real-time guidance to the user based on the analysis results.

6. The system according to claim 1, wherein the feedback unit provides support tailored to the needs of general consumers, fitness gyms, and personal trainers.

7. The system according to claim 1, wherein the tracking unit estimates the user's emotion and adjusts the tracking accuracy based on the estimated emotion.

8. The system according to claim 1, wherein the tracking unit optimizes the tracking algorithm by referring to the user's past motion data during tracking.
